(19) United States
(12) Patent Application Publication
(10) Pub. No.: US 2026/0053740 A1
(43) Pub. Date: Feb. 26, 2026

(54) PREPARATION FOR BIONIC INJECTABLE POLYPEPTIDE HYDROGEL AND USE THEREOF

(71) Applicant: NANJING CARMACURE BIOTECH CO., LTD., Nanjing, Jiangsu (CN)

(72) Inventors: Xinyi JIANG, Jinan (CN); Chen CHEN, Jinan (CN); Weiqiang JING, Jinan (CN)

(21) Appl. No.: 18/877,508

(22) PCT Filed: May 6, 2023
(86) PCT No.: PCT/CN2023/092556

§ 371 Date: Jan. 10, 2025

(30) Foreign Application Priority Data
Jun. 23, 2022 (CN) 2022107188590

(51) Int. Cl. A61K 9/06; A61K 9/00; A61K 47/42

(52) U.S. Cl. CPC A61K 9/06; A61K 9/0019; A61K 47/42

(57) ABSTRACT

A method for preparing a bionic injectable polypeptide hydrogel is provided. The hydrogel is formed using a brain extracellular matrix laminin-derived peptide DDIKVAV modified with 9-fluorenylmethoxycarbonyl (Fmoc) (Fmoc-DDIKVAV) and an immunostimulatory peptide FTKPRF modified with Fmoc (Fmoc-FTKPRF) as hydrogel monomers. These monomers further utilize non-covalent bond forces such as hydrogen bonds, hydrophobic interactions, and x-x stacking to co-assemble into a hydrogel within a short time at 37° C. The bionic hybrid polypeptide hydrogel is injectable and can serve as a drug reservoir implanted into cavities formed after tumor surgery. The drugs loaded in the hydrogel are slowly released at a stable and controlled rate and appropriate concentration within the post-surgical cavity, thereby effectively killing residual tumor cells while avoiding the toxic side effects of systemic drug administration.

The present invention claims priority to Chinese Patent Application No. 202210718859.0, filed on Jun. 23, 2022, entitled “PREPARATION FOR BIONIC INJECTABLE POLYPEPTIDE HYDROGEL AND USE THEREOF”, filed with the China National Intellectual Property Administration (CNIPA), the entire contents of which are incorporated herein by reference.

### TECHNICAL FIELD

The present invention relates to the field of biomedical technology, specifically to a method for preparing an intracavitary injectable polypeptide hydrogel and its application in post-surgical tumor treatment.

### BACKGROUND

The disclosure of the background art section is intended solely to enhance the understanding of the general background of the present invention and should not necessarily be regarded as an admission or implied acknowledgment that the information constitutes prior art known to those skilled in the art.

Malignant tumors have become one of the major social problems threatening public health due to their rapid progression, high mortality rate, and low five-year survival rate. Currently, surgical resection is commonly used in the clinical treatment of brain tumors and other tumors with brain metastases: bone tumors and other tumors with bone metastases: and melanomas. The procedure typically involves partial or complete excision of the primary or metastatic lesions to reduce tumor burden, alleviate clinical symptoms, and create conditions for subsequent radiotherapy and chemotherapy.

Glioma is the most common tumor of the central nervous system, and due to the unique characteristics and complexity of its lesion sites, it remains one of the most challenging issues in tumor treatment. Among gliomas, astrocytomas account for 70-80%. Glioblastoma Multiform (GBM) is the most malignant form of astrocytoma. The five-year survival rate for GBM does not exceed 10%. Since GBM grows in an infiltrative manner without clear boundaries with normal tissue, GBM invasive foci are detectable in normal brain tissue surrounding the tumor. These tumors often grow in critical areas of the brain, such as the basal ganglia, central sulcus, thalamus, and brainstem, making complete surgical resection difficult and leading to a high recurrence rate post-surgery: Post-operative adjuvant radiotherapy and chemotherapy have been proven to be ineffective for malignant gliomas both theoretically and practically; and their side effects are significant, often leading to a marked decline in patient's quality of life.

Brain metastasis is one of the common causes of treatment failure in malignant tumors and is typically difficult to manage clinically. The number of patients with metastatic brain tumors is approximately ten times that of patients with primary brain tumors. The median survival time for patients with brain metastases is about 5 months. For the treatment of brain metastases, surgery is the first choice for tumors with significant space-occupying effects. Post-operative treatments may include systemic chemotherapy, targeted therapy, immunotherapy, and radiotherapy. For patients with tumors that are difficult to surgically resect completely, a stereotactic biopsy under neuro-navigation is performed to obtain pathological results, followed by comprehensive treatment based on the pathology.

Bone tumors are a diverse category of diseases and are classified into primary and metastatic bone tumors. Primary bone tumors are malignant bone tumors or bone-like tissue tumors directly formed by uncontrolled growth of tumor cells. Approximately 20-34% of primary malignant bone tumors are osteosarcomas, which rank as the third most common malignancy in children and adolescents. Although adjuvant chemotherapy since the 1970s has increased the 10-year survival rate from 30% to 50%, prognosis remains unsatisfactory. Furthermore, since the 1990s, the ten-year survival rate has plateaued without further improvement. Metastatic bone tumors are more common in clinical practice. Bone metastasis occurs as a complication in 65-80% of patients with advanced breast cancer and prostate cancer. Though bone metastasis in advanced thyroid cancer, lung cancer, and kidney cancer is somewhat less common, the occurrence rate is still as high as 35-42%. As the survival duration of cancer patients increases, the incidence of bone metastasis also steadily rises. Metastatic bone tumors resulting from cancer bone metastasis are typically difficult to cure and, as the condition progresses, can lead to complications such as pathological fractures, hypercalcemia, and nerve compression, causing significant suffering for patients.

Cutaneous melanoma is a highly invasive cancer with increasing global incidence rates, characterized by early metastasis and poor prognosis, with a median survival time of only 8-9 months and a three-year survival rate of merely 10-15%. The standard treatment protocol for melanoma patients primarily involves lesion resection surgery with adjuvant radiotherapy and chemotherapy. However, melanoma shows extremely low sensitivity to radiotherapy and chemotherapy and has high recurrence and metastasis rates.

For conventional antitumor drugs administered systemically through oral or intravenous routes, only a small portion reaches the tumor site through circulation to achieve the goal of killing residual tumor cells post-surgery; while most drugs are absorbed by normal tissues before reaching the tumor tissue. Systemic drug administration results in low utilization of antitumor drugs and toxic side effects on normal tissues. Additionally, the frequent dosing required in post-operative chemotherapy can lead to drug resistance in tumor cells, resulting in poor prognosis. Compared to post-operative systemic drug administration, local drug delivery enables precise drug administration, increases drug concentration at tumor sites, and significantly reduces systemic toxicity. Among local delivery carriers, hydrogels are one of the most common, formed by crosslinking of polymer monomers into highly water-absorbent materials. Implanting drug-loaded hydrogels in post-surgical cavities offers several advantages: (1) enables local drug delivery in post-surgical cavities with low systemic toxicity: (2) can load multiple drugs for combination therapy: and (3) allows for sustained and controlled release of drug molecules at stable rates and appropriate concentrations within post-surgical cavities. In recent years, biomimetic self-assembling peptide hydrogels have received widespread attention and research interest.

Self-assembling peptide hydrogels are primarily composed of natural amino acids found in living organisms, contain no toxic chemicals, and are biodegradable. Upon degradation in vivo, the amino acids are metabolized by host cells, thus avoiding adverse effects on host cells. Over the past two decades, peptide sequence-based nanogel materials have attracted extensive attention from researchers due to their ease of synthesis, excellent gelation capabilities, and good biocompatibility and bioactivity. Hydrogels formed by self-assembling peptide systems demonstrate good biocompatibility, lack immunogenicity; do not form thrombi, and can be injected into specific tissues for local therapy, making them ideal biomaterials for nanomedicine with promising biomedical applications in tissue engineering, drug delivery, biosensors, antimicrobial drugs, and bioimaging.

### SUMMARY

To solve the above problems, one objective of the present invention is to provide a method for preparing a bionic hybrid injectable polypeptide hydrogel.

Another objective of the present invention is to provide an intracavitary injectable nanocarrier hydrogel superstructure and its application in local drug delivery for post-surgical tumors.

To achieve the above objectives, the present invention adopts the following technical solutions:

In a first aspect, the present invention provides a method for preparing a bionic hybrid injectable polypeptide hydrogel, comprising:
dissolving Fmoc-DDIKVAV and Fmoc-FTKPRF peptides in a buffer solution to obtain a mixed solution I:
adding NaOH solution to the mixed solution I to completely dissolve solid particles, obtaining a solution II:
adjusting the solution II to neutral pH to initiate self-assembly (co-assembly), thereby obtaining the bionic hybrid injectable polypeptide hydrogel.

The present invention uses a brain extracellular matrix (ECM) laminin-derived peptide DDIKVAV modified with 9-fluorenylmethoxycarbonyl (Fmoc) (i.e., Fmoc-DDIKVAV) and an immunostimulatory peptide FTKPRF modified with Fmoc (i.e., Fmoc-FTKPRF) as hydrogel monomers. These monomers further utilize non-covalent bond forces such as hydrogen bonds, hydrophobic interactions, and π-π stacking to co-assemble into a bionic hybrid injectable polypeptide hydrogel within a short time at 37° C. The polypeptide hydrogel is injectable and can serve as a drug reservoir implanted into cavities formed after tumor surgery. The drugs loaded in the hydrogel are slowly released at a stable and controlled rate and appropriate concentration within the post-surgical cavity, thereby effectively killing residual tumor cells while avoiding the toxic side effects of systemic drug administration.

In a second aspect, the present invention provides a bionic hybrid injectable polypeptide hydrogel prepared by the above method.

In a third aspect, the present invention provides an application of the bionic hybrid injectable polypeptide hydrogel prepared as described for the preparation of drugs for post-surgical tumor treatment or as a local drug delivery system for post-surgical cavities.

### Advantageous Effects of the Invention

(1) The co-assembling polypeptide hydrogel of the present invention is injectable and can serve as a drug reservoir implanted into cavities formed after tumor surgery. The drugs loaded in the hydrogel are slowly released at a stable and controlled rate and appropriate concentration within the post-surgical cavity, thereby effectively killing residual tumor cells while avoiding the toxic side effects of systemic drug administration.

(2) The method of preparation in the present invention is simple and practical, making it easy to implement widely.

### BRIEF DESCRIPTION OF THE DRAWINGS

The accompanying drawings to the specification, which form part of the present invention, are used to provide a further understanding of the present invention, and the illustrative examples of the present invention and the description thereof are used to explain the present invention and are not unduly limiting the present invention.

FIG. 1 shows a macroscopic image of gelation of the bionic hybrid polypeptide hydrogel prepared in Example 1 of the present invention.

FIG. 2 shows characterization of injectable properties of the bionic hybrid polypeptide hydrogel prepared in Example 1 of the present invention.

FIG. 3 shows a transmission electron microscopy (TEM) image of the bionic hybrid polypeptide hydrogel prepared in Example 1 of the present invention: scale bar: 200 μm.

FIG. 4 shows a scanning electron microscopy (SEM) image of lyophilized powder of the bionic hybrid polypeptide hydrogel prepared in Example 1 of the present invention: scale bar: 500 μm.

FIG. 5 shows modulus analysis of the bionic hybrid polypeptide hydrogel prepared in Example 1 of the present invention.

FIG. 6 shows viscosity analysis of the bionic hybrid polypeptide hydrogel prepared in Example 1 of the present invention.

### DESCRIPTION OF THE PREFERRED EMBODIMENTS

It should be noted that the following detailed description is illustrative and intended to provide further clarification of the present invention. Unless otherwise specified, all technical and scientific terms used in the present invention have the same meaning as commonly understood by those of ordinary skill in the art to which the present invention belongs.

A method for preparing a bionic hybrid injectable polypeptide hydrogel comprises the following steps:
(1) dissolving Fmoc-DDIKVAV and Fmoc-FTKPRF peptides in PBS buffer solution to obtain a mixed solution I;
(2) under magnetic stirring, adding NaOH solution dropwise to the mixed solution I until undissolved solid particles are completely dissolved, obtaining a solution II;
(3) adding HCl solution dropwise to the solution II, adjusting to neutral pH, and thoroughly mixing using a vortex mixer at room temperature to obtain the bionic hybrid injectable polypeptide hydrogel.

In some embodiments, a mass ratio of Fmoc-DDIKVAV to Fmoc-FTKPRF peptides is (1-1.5): (1-1.5).

In some embodiments, the PBS buffer solution has a pH of 7.2-7.4.

In some embodiments, a mass to volume ratio of Fmoc-DDIKVAV to the PBS buffer solution is 1 mg: (20-25) μL.

In some embodiments, a concentration of NaOH solution is 0.25-0.3 M.

In some embodiments, the NaOH solution is added dropwise under magnetic stirring at a stirring speed of 50-80 rpm min⁻¹.

In some embodiments, pH is adjusted using HCl solution with a concentration of 0.1-0.2 M.

In some embodiments, the co-assembly is performed under vortex stirring for 1-2 minutes.

The present invention is described in further detail below in connection with specific embodiments which should be noted as an interpretation of the present invention and not as a limitation.

In the following examples, Fmoc-DDIKVAV (5 mg) and Fmoc-FTKPRF (5 mg) peptides were synthesized by a commercial company. Their structures are shown below:

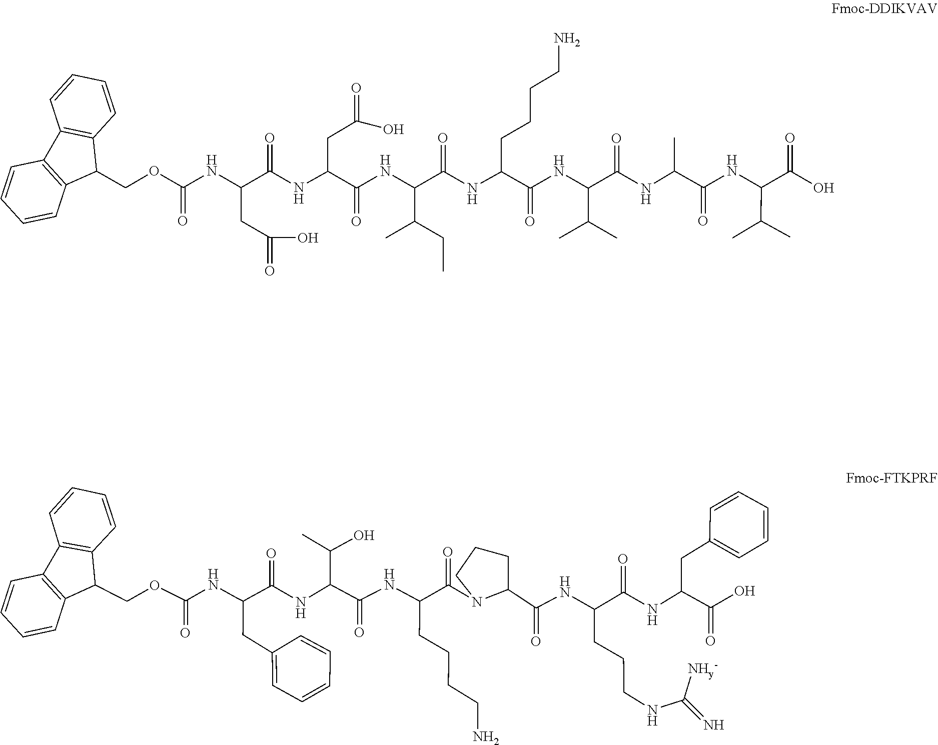

### Example 1: Method for Preparing Bionic Hybrid Polypeptide Hydrogel

(1) A clean, dry 1 mL EP tube was used. Fmoc-DDIKVAV (5 mg) and Fmoc-FTKPRF (5 mg) peptides were weighed separately, followed by addition of 100 μL of 0.25 M NaOH phosphate buffer solution. The mixture was dissolved and a magnetic stir bar was added.

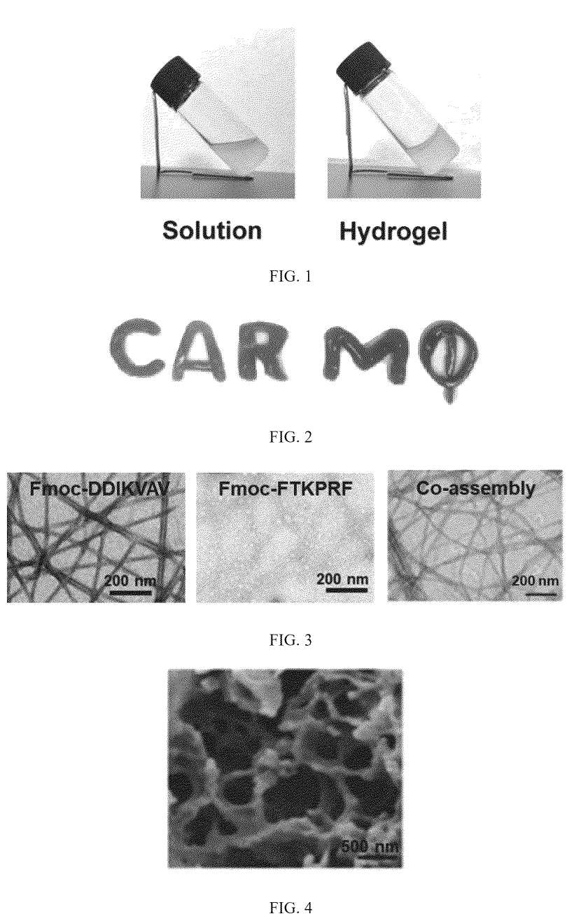

(2) A clean, dry 10 mL beaker was used. A precise amount of concentrated hydrochloric acid was weighed and added to double-distilled water, stirred with a glass rod to achieve a final concentration of 0.1 M. HCl solution was added dropwise to the above 1 mL EP tube, and the pH of the mixed solution was monitored using a pH meter. The pH of the mixed solution was adjusted to 7.2, and the EP tube was vortexed for 2 minutes. The peptide units further co-assembled into a hydrogel through non-covalent bond forces including hydrogen bonds, hydrophobic interactions, and x-x stacking. The macroscopic image of gelation of the bionic hybrid polypeptide hydrogel prepared in the example and blank solution control are shown in FIG. 1.

### Example 2: Investigation of Injectable Properties of Bionic Hybrid Polypeptide Hydrogel

The pre-assembly mixed solution (containing Fmoc-DDIKVAV, Fmoc-FTKPRF, NaOH, and HCl) and rhodamine B solution were injected through a 25-gauge needle into a mold containing CAR-MΦ prior to incubation, and the hydrogel shape was observed after removing the mold. Results showed that the bionic hybrid polypeptide hydrogel of the present invention possessed injectable properties. As shown in FIG. 2.

### Example 3: Microscopic Structure Investigation of Bionic Hybrid Polypeptide Hydrogel

The prepared Fmoc-DDIKVAV hydrogel, Fmoc-FTKPRF hydrogel, and Fmoc-DDIKVAV-Fmoc-FTKPRF hybrid hydrogel were diluted with ultrapure water. Samples with a total volume of 10 μL were deposited on fresh copper grids, removed, air-dried, and imaged using transmission electron microscopy. Results showed that Fmoc-DDIKVAV hydrogel, Fmoc-FTKPRF hydrogel, and Fmoc-DDIKVAV-Fmoc-FTKPRF hybrid hydrogel possessed interconnected nanofiber network structures. As shown in FIG. 3.

The internal microstructure of the bionic hybrid polypeptide hydrogel was observed using Scanning Electron Microscope (SEM). After gold-sputtering the lyophilized bionic hybrid polypeptide hydrogel, the morphology of the hydrogel was observed by field emission scanning electron microscopy at an accelerating voltage of 5 kV. Results showed that the polypeptide hydrogel had a network structure with uniform porosity, as shown in FIG. 4.

### Example 4: Rheological Investigation of Bionic Hybrid Polypeptide Hydrogel

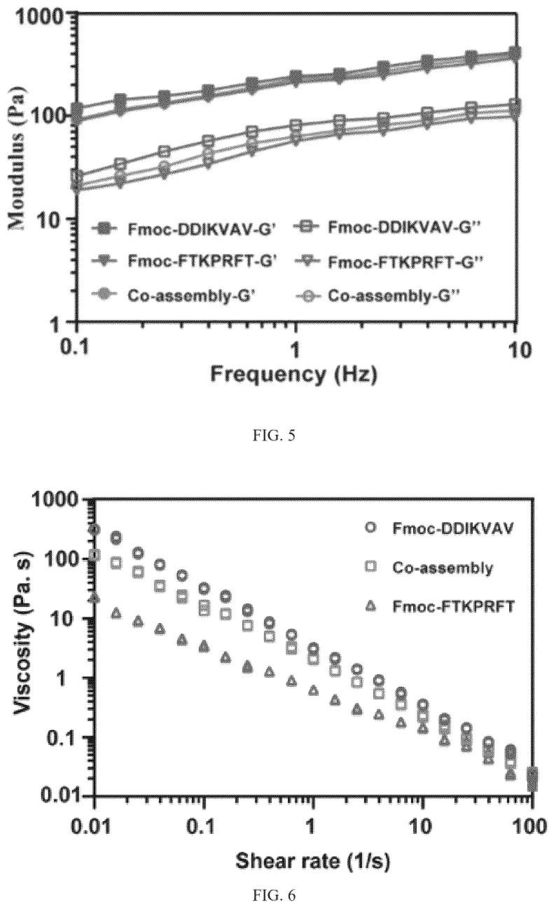

The modulus of the hydrogel was analyzed using a rheometer (Anton-Paar MCR302). The polypeptide hydrogel was placed on parallel plates and measured at 37+0.1° C. Frequency sweep rheological analysis of the polypeptide hydrogel was performed within a frequency range of 0.1-100rad s⁻¹, recording curves of storage modulus (G′) and loss modulus (G″) changes with frequency for different hydrogels. Results showed that both G′ and G″ increased with increasing frequency, and the storage modulus was greater than the loss modulus, indicating that the polypeptide hydrogel could maintain high elasticity. As shown in FIG. 5.

A rheometer was used to analyze the viscosity and shear stress of the hydrogel. Results showed that the viscosity of all three hydrogels decreased with increasing shear rate, indicating that the bionic polypeptide hydrogel possessed shear-thinning capability, further demonstrating its injectable properties.

The above descriptions are merely preferred embodiments of the present invention and are not intended to limit the present invention. For those skilled in the art, various changes and modifications can be made to the present invention. Any modification, equivalent replacement, or improvement made within the spirit and principles of the present invention should be included within the scope of the present invention's protection.

1. A method for preparing a bionic hybrid injectable polypeptide hydrogel, comprising:
dissolving Fmoc-DDIKVAV and Fmoc-FTKPRF peptides in a buffer solution to obtain a mixed solution I;
adding NaOH solution to the mixed solution I to completely dissolve solid particles, obtaining a solution II;
adjusting the solution II to neutral pH to initiate co-assembly, thereby obtaining the bionic hybrid injectable polypeptide hydrogel.

2. The method according to claim 1, wherein a mass ratio of Fmoc-DDIKVAV to Fmoc-FTKPRF peptides is (1-1.5):(1-1.5).

3. The method according to claim 1, wherein the buffer solution is PBS buffer solution with a pH of 7.2-7.4.

4. The method according to claim 1, wherein a mass to volume ratio of Fmoc-DDIKVAV to buffer solution is 1 mg: (20-25) μL.

5. The method according to claim 1, wherein a concentration of NaOH solution is 0.25-0.3 M.

6. The method according to claim 1, wherein NaOH solution is added dropwise under magnetic stirring at a stirring speed of 50-80 rpm min⁻¹.

7. The method according to claim 1, wherein pH is adjusted using HCl solution with a concentration of 0.1-0.2 M.

8. The method according to claim 1, wherein the co-assembly is performed under vortex stirring for 1-2 minutes.

9. A bionic hybrid injectable polypeptide hydrogel prepared by the method according to claim 1.

10. The bionic hybrid injectable polypeptide hydrogel according to claim 9, being used for preparing drugs for post-surgical tumor treatment or a local drug delivery system for post-surgical cavities.
